(19) United States
(12) Patent Application Publication
(10) Pub. No.: US 2003/0013740 A1
(43) Pub. Date: Jan. 16, 2003

(54) STABLE DOSAGE FORMS OF FLUOXETINE AND ITS ENANTIOMERS

(76) Inventors: MARTIN P. REDMON, MARLBOROUGH, MA (US); HAL T. BUTLER, MARLBOROUGH, MA (US); STEPHEN A. WALD, MARLBOROUGH, MA (US)

Correspondence Address: PENNIE & EDMONDS, 1155 AVENUE OF THE AMERICAS, NEW YORK, NY, 100362711

(21) Appl. No.: 09/049,227

(22) Filed: Mar. 27, 1998

(51) Int. Cl. A61K 31/445

(52) U.S. Cl. 514/330

(57) ABSTRACT

Chemically and physically stable pharmaceutical formulations, of the potent antidepressant, fluoxetine, its enantiomers and salts.

### 1. FIELD OF THE INVENTION

The present invention relates to chemically and physically stable pharmaceutical compositions containing fluoxetine or an enantiomer or salt thereof.

### 2. BACKGROUND OF THE INVENTION

Many factors affect the stability of a pharmaceutical product, including the stability of the therapeutic drug ingredient(s), the potential interaction between the therapeutic drug ingredient(s) and the inactive ingredient(s), the manufacturing process, the packaging, the environmental conditions encountered during shipment, storage and handling, the length of time between manufacture and usage and the type of the dosage form. In addition to physical stability, the chemical stability of the pharmaceutical product should be considered. Knowledge of the physical and chemical stability of a pharmaceutical formulation is very important for at least three primary reasons.

First, a pharmaceutical product, preferably, should appear fresh, elegant and professional. Any changes in physical appearance and color including fading, color variation, appearance of haziness and the like can cause the patient to lose confidence in the product. Second, since some products are dispensed in multiple-dose containers, uniform dosage of the therapeutic agent(s) over time must be assured. For example, a non-uniform dosage pattern may be indicated by a cloudy solution, a broken emulsion, a discolored tablet, a discolored capsule or the like. Third, the therapeutic drug ingredient(s) must be available to the patient throughout the expected shelf life of the dosage form. A breakdown in the physical or chemical integrity of the dosage form can lead to a lack of bioavailability or detrimentally altered bioavailability of the therapeutic drug ingredient(s).

A variety of dosage forms are available for administering drugs; for example, troches, tablets and capsules, which typically, contain the drug ingredient, a diluent and other excipients such as lubricants and the like are well known in the art. Well known excipients include, for example, coating agents, colorants, desiccants, emulsifying agents, solubilizing agents, flavors, anti-caking agents, plasticizers, suspending agents, viscosity increasing agents, binders, diluents, wetting agents and the like.

Lactose is a commonly used diluent or excipient. Spray-dried lactose is a commonly available form of lactose. Since the advent of spray-dried lactose, its use as an excipient has expanded. The rapid acceptance of spray-dried lactose is, in part, due to its ease of incorporation in direct compression tablets. In this application, spray-dried lactose is in its ready-to-use form and does not require further granulation or introduction of complicated processing steps. Spray-dried lactose can also be readily and conveniently incorporated into a troche or a capsule dosage form. Spray-dried lactose may be directly added to a drug to yield a desired dilution ratio therewith. Thereafter, for example, the combination of the lactose and the drug may be dry compressed into a tablet or formulated into a troche or a capsule with other excipients, as necessary.

Lactose, whether spray-dried or not, is typically present in equilibrium between its alpha and beta forms, wherein interconversion between these forms is ongoing. Alpha-lactose is a disaccharide of beta-D-galactose and alpha-D-glucose. Beta-lactose is a disaccharide of beta-D-galactose and beta-D-glucose. Beta-lactose occurs only in its anhydrous form, whereas alpha-lactose may be obtained either in anhydrous form or as a monohydrate.

During interconversion between the alpha and beta forms of lactose, an aldehyde intermediate is formed which is known to be incompatible with most primary amines. Primary amines add to the carbonyl carbon of aldehydes (and ketones) to form imines:

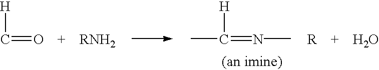

The incompatibility of most primary amines with lactose is well-recognized. See, Castello et al., J. Pharm. Sci., 51 (2):106-108 (February 1962). See also, Blaug et al., J. Pharm. Sci., 61(11):1770-1775 (November 1972); Hartauer et al., Drug Dev. and Indust. Pharm., 17(4):617-630 (1991).

Castello et al. tested the compatibility of amphetamine sulfate (a primary amine salt) with lactose. They found that a mixture of lactose and amphetamine sulfate discolored, especially in the presence of alkaline lubricants such as magnesium stearate. Blaug et al. tested dextroamphetamine sulfate (a primary amine salt) with spray-dried lactose. They found that the lactose formed a Schiff base (i.e., an imine) in the presence of dextroamphetamine sulfate. Hartauer et al. tested aminophylline with lactose, and found that some incompatibility, evidenced by discoloration, between aminophylline and lactose occurred, especially when heat, of about 60° C., was applied. Aminophylline contains a ratio of two molecules of theophylline (a secondary amine) for one molecule of ethylene diamine (a primary amine). However, Hartauer et al. tested these components and found that while theophylline alone (a secondary amine) did not react with lactose in the presence or absence of heating to 60° C., ethylene diamine did react with the lactose, especially when heated to 60° C. Thus, the incompatibility of aminophylline with lactose appeared to result from incompatibility of the primary amine component of aminophylline, ethylenediamine, with lactose.

The prescription drug fluoxetine hydrochloride or PROZAC®, a secondary amine, was believed to be compatible with lactose, as several patent publications have described lactose as a suitable excipient and several generic versions of fluoxetine hydrochloride are formulated with lactose. However, the commercially available product, PROZAC®, is available as a Pulvule® dosage form that does not contain lactose. According to the Physician's Desk Reference, 52nd Edition, Medical Economics Co., Montvale, N.J., p. 1293 (1998), each unit dosage form of solid PROZAC® contains 10 or 20 mgs of fluoxetine hydrochloride, FD&C Blue No. 1, gelatin, iron oxide, silicone, starch, titanium dioxide and other active ingredients. Fluoxetine is also available in an oral solution. In either case, the commercial available forms are thought to be stable. See, e.g., Petterson et al., Am. J. Hosp. Pharm. 51:1342 (1994)

U.S. Pat. Nos. 5,104,899, 5,589,511, 5,648,396 and 5,708,035 all relate to the preparation and use of pharmaceutical compositions containing optically pure S(+) or R(−) fluoxetine. Each of these patents reports lactose as an acceptable excipient for use with the enantiomers of fluoxetine. Similarly, U.S. Pat. No. 5,356,934 discloses lactose as an acceptable ingredient for use with (R)-fluoxetine. In addition, U.S. Pat. Nos. 4,683,235 and 4,594,358 mention lactose as an acceptable excipient for racemic fluoxetine compositions.

EP 0,693,281 A2 discloses dispersable tablets (i.e., tablets that rapidly dissolve or disperse in water and are intended to be ingested after dispersal) containing fluoxetine hydrochloride. These dispersable tablets include lactose as an excipient. For example, a dispersable tablet containing fluoxetine hydrochloride, sodium starch glycolate, lactose, L-HPC 21, sodium saccharin and mint aroma is described in Example 1 of this patent document.

Contrary to these publications, and contrary to the use of fluoxetine and lactose by generic pharmaceutical companies, it has been discovered that certain secondary amine containing drugs, including racemic fluoxetine, its enantiomers and salts thereof, are thermally and chemically unstable in the presence of lactose. It has also been discovered that water can accelerate the degradation of fluoxetine in the presence of lactose or related compounds.

Thus, the present invention provides stable solid pharmaceutical dosage forms that have the required attributes of a dosage form without the thermal or chemical instability that occurs with the use of lactose.

It is desirable to prepare stable solid pharmaceutical formulations of fluoxetine, its enantiomers or salts that avoid the incompatibility between drugs containing secondary amines and excipients such as lactose and the like.

### 3. SUMMARY OF THE INVENTION

The present invention encompasses stable solid pharmaceutical dosage forms of fluoxetine, its enantiomers or salts, preferably acid addition salts. The dosage forms are physically and chemically stable, high performance compositions which avoid any incompatibility between the active secondary amine containing compounds, such as the fluoxetine active ingredient, and certain excipients that are substrates for the Maillard reaction including but not limited to lactose. The preferred dosage forms are compressed tablets. These compressed tablets are preferably lactose-free. The most preferred dosage forms are lactose-free or non-hygroscopic.

The present invention also relates to a lactose-free pharmaceutical composition which includes fluoxetine or its enantiomers, or a pharmaceutically acceptable salt thereof, and at least one non-lactose pharmaceutically acceptable excipient. In another embodiment, the invention relates to a solid pharmaceutical composition which includes fluoxetine or its enantiomers, or a pharmaceutically acceptable salt thereof, and at least one pharmaceutically acceptable excipient, wherein said excipient is not lactose.

In one embodiment, at least one non-lactose pharmaceutically acceptable excipient is a binder, a filler, or mixtures thereof. In another embodiment, at least one pharmaceutical excipient is a binder, a filler, or mixtures thereof. In a preferred embodiment, the above excipients further include a lubricant, a disintegrant, or mixtures thereof. In a preferred embodiment, the pharmaceutical composition is substantially free of all mono- or di-saccharide excipients. In another embodiment, the pharmaceutical composition is substantially free of all mono- or di-saccharide fillers.

The invention also relates to a thermally stable solid pharmaceutical composition free of lactose which comprises fluoxetine, an optically pure enantiomer thereof, or a pharmaceutically acceptable salt thereof, and at least one pharmaceutically acceptable excipient. The invention also relates to a chemically stable solid pharmaceutical composition free of lactose which includes about 1% to about 99% by weight of fluoxetine, an optically pure enantiomer, or a pharmaceutically acceptable salt thereof, and about 99% to about 10% by weight of at least one pharmaceutically acceptable excipient.

In one embodiment, fluoxetine, or its enantiomers or salts, is present in an amount from about 1 mg to about 200 mg. In a more preferred embodiment, fluoxetine, or its enantiomers or salts, is present in an amount of about 10 mg to about 80 mg. In another preferred embodiment, fluoxetine, its enantiomers or salts, is present in a therapeutically effective amount for treatment of depression, an obsessive-compulsive disorder, anxiety or obesity. In yet another preferred embodiment, the therapeutically effective amount is sufficient for the prophylaxis or treatment in humans of depression or migraine headache.

The invention also relates to a solid pharmaceutical composition that includes fluoxetine, its enantiomers or a pharmaceutically acceptable salt thereof, microcrystalline cellulose and pre-gelatinized starch. In one embodiment, the solid pharmaceutical composition is provided in a tablet or a capsule dosage form, preferably a compressed tablet.

The invention also relates to a method for treating disorders in a mammal by administering a therapeutically effective amount of one of the compositions of the invention. In a preferred embodiment, the mammal is a human. The disorders include depression, anxiety, obsessive-compulsive disorder, bulimia, obesity, migraine headache and anxiety.

### 4. DETAILED DESCRIPTION OF THE INVENTION

Based upon the pharmacological benefits of fluoxetine, its enantiomers and salts, there is need for stable high performance dosage forms of these active ingredients. In particular, there is a need for a solid tablet form, particularly a stable compressed tablet. The inventors have found that by eliminating lactose and using the alternative ingredients described herein, lactose-free dosage forms of fluoxetine, its enantiomers or salts are surprisingly chemically and physically stable. This stability may be achieved by the present invention without loss of either manufacturing ease or dosage performance.

The present invention is directed to chemically and physically stable pharmaceutical formulations which include fluoxetine, an enantiomer, or a pharmaceutically acceptable salt thereof, and a pharmaceutically acceptable carrier or excipient that does not include or utilize any form of lactose. Lactose has been widely accepted and used by the pharmaceutical industry, inter alia, because of its ease of manufacture. However, applicants have advantageously found that formulations containing secondary amine drugs (i.e., compounds having a secondary amine moiety) and lactose are unstable over time and degrade more rapidly upon exposure to heat and moisture.

Secondary amines were previously considered to be compatible with lactose, especially at ambient temperatures or where exposure to heat (e.g., below about 60° C.) is either minimal or altogether avoided. As noted, fluoxetine as well as its enantiomers and salts, although available as a lactose-free Pulvule® capsule, have been described as being compatible with lactose. Only after the present invention has it been reported that fluoxetine may interact adversely with lactose. Wirth et al., J. Pharm. Sci., 87(1):31 (January 1998).

It has been discovered that physical and/or chemical incompatibility exists between the secondary amine, fluoxetine, its enantiomers and salts, and lactose. Without being limited by theory, it is believed that the incompatibility of fluoxetine, its enantiomers and salts with lactose results from the formation of enamines due to reaction between the aldehyde intermediate of lactose and a secondary amine:

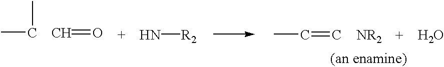

It has also been discovered that the incompatibility exists even at ambient temperatures (e.g., temperatures below about 60° C.) and at ambient relative humidity. Further, Applicants have also discovered highly stable pharmaceutical compositions containing fluoxetine or its enantiomers without the use of the widely accepted excipient lactose.

According to the present invention, fluoxetine, or an enantiomer or salt thereof is provided in lactose-free pharmaceutical composition. These compositions possess potent activity as selective serotonin reuptake inhibitors and are useful in treating a variety of conditions. Some of these conditions include, for example, depression, obesity, migraine headache, obsessive-compulsion disorder, anxiety, bulimia and related disorders.

More importantly, these lactose-free compositions provide a stable and convenient dosage form for delivering fluoxetine, its enantiomers or salts to humans. The lactose-free compositions of the invention are stable, inter alia, in that they have significant shelf-life. Further, the compositions of the invention remain stable even when exposed to mild temperature and humidity changes. Moreover, even though the compositions of the invention are lactose-free, the compositions are still easily manufactured, and the compositions have desirable dosage performance properties. The compositions of the invention include solid unit dose formulations comprising fluoxetine, an optically pure enantiomer, or a pharmaceutically acceptable salt thereof, and at least one non-lactose pharmaceutically acceptable excipient. The compositions may also optionally include other therapeutic ingredients, binders/fillers, disintegrants, lubricants, anti-caking agents, preservatives, film coating agents, sweetening agents, colorants, flavors, desiccants, plasticizers, dyes, dispersing agents and/or surface active agents. However, any such optional ingredient must be compatible with fluoxetine or its enantiomers, a secondary amine, to insure the stability of the formulation.

It is preferred that the lactose-free dosage form of fluoxetine, an enantiomer or salt thereof, made in accordance with the present invention comprise the active ingredient and at least one non-lactose excipient. Examples of such excipients are well known in the art and are listed in the USP (XXI)/NF (XVI), incorporated herein in its entirety by reference thereto. It is further preferred that the lactose-free fluoxetine dosage forms made in accordance with the present invention comprise fluoxetine, an enantiomer or salt thereof, a binder/filler and a lubricant in pharmaceutically compatible and pharmaceutically acceptable amounts. It is even further preferred that the lactose-free fluoxetine dosage forms made in accordance with the present invention comprise fluoxetine, or an enantiomer or salt thereof, microcrystalline cellulose, pre-gelatinized starch, and magnesium stearate.

Other sugars, such as fructose and sucrose, as well as carbohydrate fillers, cause or may cause, similar, although not as severe, degradation as that caused by lactose when used in combination with fluoxetine containing formulations. Thus, in another embodiment, the lactose-free pharmaceutical compositions comprise fluoxetine, an enantiomer or a pharmaceutically acceptable salt thereof, and at least one non-lactose pharmaceutically acceptable excipient, and do not contain any mono- or disaccharide excipients, including, but not limited to, glucose, sucrose, and fructose.

As mentioned above, fluoxetine formulations containing lactose that are exposed to unbound water, e.g., moisture or humidity, degrade more rapidly. The addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80.

Further, the effect of water on a formulation is of great significance since conditions favorable for hygroscopicity, e.g., moisture and/or humidity, are commonly encountered during manufacture, handling, packaging, storage, shipment and use of the formulation. Thus, it is clear that the use of lactose in pharmaceutical compositions or formulations containing an active form of fluoxetine should be avoided due to the substantial contact with moisture and/or humidity that the compositions have under normal manufacturing, packaging and storage conditions.

Moreover, although excipients other than lactose may be readily used to manufacture the disclosed lactose-free pharmaceutical compositions of fluoxetine without affecting the manufacturability and therapeutic performance of the compositions, spray-dried lactose continues to be an excipient of choice. In the spray-dried form, lactose is among the best of all direct compression fillers in fluidity and is very effective for low dose formulations (e.g., ≦50 mg per dose) where the compactibility of the active ingredient does not play a major role in the formulation. See, e.g., R. Shangraw, Selection of Manufacturing Process nd Excipients with an Emphasis on Direct Compression, Course material from Granulation, Tableting, and Capsule Technology, Center for Professional Advancement, East Brunswick, N.J., 1996. Therefore, when possible, it is desirable to include lactose among the available possible excipients for the solid dosage forms or pharmaceutical composition of fluoxetine.

Therefore, as an alternative, the present invention encompasses physically and chemically stable pharmaceutical compositions, particularly, solid pharmaceutical formulations, which comprise fluoxetine and an optically pure enantiomer, or a pharmaceutically acceptable salt thereof, and optionally one or more pharmaceutically acceptable excipients, including but not limited to lactose, wherein the lactose containing formulations are anhydrous, i.e., substantially free of unbound water. The invention further encompasses thermally and chemically stable non-hygroscopic pharmaceutical compositions which comprise an active form of fluoxetine, or an enantiomer or a pharmaceutically acceptable salt thereof, and one or more excipients or ingredients including, but not limited to, lactose. Without being limited by any theory, these stable anhydrous or non-hygroscopic pharmaceutical compositions are based, in part, on applicants' discovery that the incompatibility between secondary amines such as fluoxetine and lactose, or other mono-or di-saccharides, is accelerated and/or possibly initiated by exposure of such formulations to unbound water.

Thus, if lactose is a desired excipient, another aspect of the invention relates to non-hygroscopic or anhydrous pharmaceutical compositions comprising an active form of fluoxetine, lactose and optionally one or more additional excipients or ingredients wherein the resulting harmaceutical compositions are substantially free of unbound water. It should be recognized that the non-hygroscopic or anhydrous formulations can be made by standard methods, provided that suitable excipients are selected such that the resulting pharmaceutical compositions are substantially free of unbound water, and processing is conducted using conditions of low humidity.

Anhydrous pharmaceutical composition prepared in accordance with the present invention should be prepared and stored such that the anhydrous nature is maintained. Accordingly, these compositions will be packaged using materials well known in the art for preventing exposure of the pharmaceutical composition to water, allowing them to be included in suitable formulary kits. Such packaging will include, but not be limited to, hermetically sealed foils, plastic or the like, unit dose containers, blister packs, or strip packs.

A second alternative aspect of the invention encompasses a method of preparing a solid pharmaceutical formulation comprising an active form of fluoxetine and lactose which method comprises admixing under anhydrous or low moisture/humidity conditions, active form of fluoxetine, or a pharmaceutically acceptable salt thereof, and lactose wherein said ingredients are substantially free of unbound water. The method may optionally further comprise packaging said anhydrous or non-hygroscopic solid formulation under low moisture conditions. By using such conditions, the risk of contact with water is reduced and the degradation of active fluoxetine is prevented or substantially reduced during processing and storage. Further, the final packaged product has little or no unbound water present which substantially improves stability and prevents degradation. Such compositions can be provided in hermetically sealed packages such as vials, sealed packets, blister packs and other vacuum sealed and moisture free containers well known to the skilled artisan.

The preferred amount of fluoxetine, or an enantiomer or salt thereof in all the dosage forms made in accordance with the present invention should be a therapeutically effective amount thereof which is also a medically acceptable amount thereof. Actual dosage levels of fluoxetine or an enantiomer thereof in the pharmaceutical compositions of the present invention may be varied so as to obtain an amount of fluoxetine which is effective to achieve the desired therapeutic response for a particular patient, and mode of administration, without being toxic to the patient.

The selected dosage level and frequency of administration will depend upon a variety of factors including the route of administration, the time of administration, the rate of excretion of the therapeutic agent(s) e.g., fluoxetine, or an enantiomer or salt thereof, the duration of the treatment, other drugs, compounds and/or materials used in combination with fluoxetine or its enantiomers, the age, sex, weight, condition, general health and prior medical history of the patient being treated and the like factors well known in the medical arts. For example, the dosage regimen is likely to vary with pregnant women, nursing mothers and children relative to healthy adults.

A physician having ordinary skill in the art can readily determine and prescribe the therapeutically effective amount of the pharmaceutical composition required. For example, the physician could start doses of fluoxetine or its enantiomers employed in the pharmaceutical composition of the present invention at levels lower than that required to achieve the desired therapeutic effect and gradually increase the dosage until the desired effect is achieved.

A suitable daily dose of fluoxetine or its enantiomers will be that amount of fluoxetine or its enantiomers which is the lowest effective dose to produce a desired therapeutic effect. Such a therapeutically effective dose will generally depend upon the factors described above. For example, the unit dose of fluoxetine or its enantiomers or salts may contain from about 1 mg to about 200 mg and preferably about 2 mg to about 100 mg. For example, unit dosages may be formulated with 1 mg, 5 mg, 10 mg, 15 mg, 20 mg, 25 mg, 30 mg, 35 mg, 40 mg, 45 mg, 50 mg, 60 mg or 80 mg of fluoxetine, or an enantiomer or salt thereof. If desired, the effective daily dose of fluoxetine or its enantiomers may be administered separately at appropriate intervals throughout the day, optionally, in unit dosage forms as two, three, four, five, six or more sub-doses. As previously noted, the preferred dosage forms are tablets, pastilles, pills, lozenges, syrups, capsules and the like. However, other pharmaceutically acceptable dosage forms such as powders, granules, dragees and the like may be used.

It is noted that all components comprising the dosage forms of fluoxetine or its enantiomers made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable provide bioavailability, stability, procedures for proper handling and storage and methods for their examination and formulas for their manufacture or preparation.

The lactose-free, non-hygroscopic or anhydrous dosage forms of fluoxetine or its enantiomers described and claimed herein meet the pharmaceutical standards set forth in the USP/NF (e.g., USP XXI/NF XVI) for each of the ingredients in pharmaceutically acceptable combinations and pharmaceutically acceptable amounts to at least meet the standards set forth in the USP XXI/NF XVI, incorporated herein in its entirety by reference thereto. In addition, it should be noted that fluoxetine, its enantiomers and salts can be made according to methods well known in the art, including those disclosed in U.S. Pat. Nos. 4,314,081, 5,104,899, 5,589,511, and 5,648,396, which are incorporated herein by reference thereto for the express purpose of teaching methods to prepare fluoxetine or its enantiomers.

Stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredient(s) (e.g., fluoxetine or its enantiomers and excipients) and the like. Physical factors such as heat, light and moisture may initiate or accelerate chemical reactions.

For convenience, certain terms employed herein are defined as follows. The term “carrier” as used herein is synonymous with the term “vehicle.” The term “lactose-free” as used herein is intended to mean that the amount of lactose present, if any, in the dosage form of fluoxetine or its enantiomers is insufficient to cause the incompatibility between fluoxetine or salts or enantiomers thereof and lactose discovered by the inventors to detrimentally affect the potency of the fluoxetine below about 90% of initial potency over the shelf life of the dosage form. The term “unbound water” as used herein means water that is not present in the form of a stable hydrate of one or more components of the pharmaceutical composition, e.g., alpha lactose monohydrate. Similarly, the term “anhydrous” as used herein means the amount of unbound water present, if any, in the dosage form is insufficient to initiate and/or accelerate the incompatibility between fluoxetine and lactose. Further, “anhydrous” or “anhydrous” conditions or nature as used herein means substantially free of unbound water including moisture. The term “non-hygroscopic” as used herein means the overall formulation is substantially non-hygroscopic, i.e., does not provide unbound water sufficient to initiate and/or accelerate the incompatibility between fluoxetine and lactose. The term “additives” is synonymous with the term “excipients” as used herein.

As used herein “fluoxetine or an enantiomer or salt thereof” means racemic fluoxetine and salts of racemic fluoxetine; optically pure (S)-fluoxetine and salts thereof, and optically pure (R)-fluoxetine and salts thereof. In other words, salts of the racemate and enantiomers are included within the invention.

As used herein the terms “optically pure”, “substantially free of the R-enantiomer”, or “substantially free of its S-enantiomer” means that the composition contains greater than 95% of the desired enantiomer by weight, preferably greater than 98% of the desired enantiomer by weight, and most preferably greater than about 99% of the desired enantiomer by weight, said percent based upon the total weight of fluoxetine. In other words, the term “substantially free” means less than about 5 weight percent, preferably less than about 2 weight percent, and more preferably less than about 1 weight percent.

As used herein, “fluoxetine” or “racemic fluoxetine” refers to the compound (±)N-methyl-3-phenyl-3-[(α,α,α-trifluoro-p-tolyl)oxy]propylamine or (±)N-methyl-3-(p-trifluormethylphenoxy)-3-phenyl propylamine, the free base, anhydrous forms, hydrated forms, solvates or clathrates thereof.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

As used herein, the term “oral administration of a solid unit dosage form” means that the dosage form is administered via the oral cavity; and that a whole pill is placed in the mouth and swallowed such that the active ingredient is not released in the mouth; or preferably that the solid unit dosage form does not begin to substantially dissolve in the mouth.

Further, the term “pharmaceutically acceptable” excipient is employed to mean that there are no untoward chemical or physical incompatibilities between fluoxetine or its enantiomers (or a salt thereof) and any of the excipient components of a given lactose-free dosage form. For example, an untoward chemical reaction is one wherein the potency of the fluoxetine or its enantiomers (or salt thereof) is detrimentally reduced or increased due to the addition of one or more excipients. Another example of an untoward chemical reaction is one wherein the taste of the fluoxetine (or an enantiomer or salt thereof) dosage form becomes excessively unpalatable. Each excipient must be “acceptable” in the sense of being compatible with the other ingredients of the lactose-free fluoxetine formulation and not injurious to the patient.

Physical incompatibility refers to incompatibility among the various components of the dosage form such as fluoxetine or its enantiomers (or salt thereof) and any of the excipient(s) thereof. For example, the combination of the excipient(s) and fluoxetine may form an excessively hygroscopic mixture or an excessively segregated mixture to the degree that the desired shape of the dosage form (e.g., tablet, troche, capsule), its stability or the like cannot be sufficiently maintained to be able to administer the dosage form in compliance with a prescribed dosage regimen as desired.

Most often, antidepressants, such as fluoxetine, are administered orally by means of solid dosage forms such as tablets, capsules, troches, caplets and the like. Further, capsule dosage forms such as hard gelatin capsules, soft gelatin capsules and the like may also be used. However, tablets remain a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives. The invention is preferably directed to compressed but non-disposable tablets.

As used herein, “dispersable tablets” refers to solid, orally administered pharmaceutical forms which must dissolve in less than three (3) minutes in water at 19° C.-21° C. and disperse evenly in water. This test involves placing two tablets in 100 ml of water and shaking them until they disperse completely. The dispersion produced by this means must pass through a screen with a nominal mesh of 710 microns. (Pharmacopea Britanica, Vol. II 1988). This test is referred to herein as the “DISSOLUTION TEST”. A tablet capable of this type of in vitro dissolution is also referred to herein as a rapid dissolving tablet. In a preferred embodiment, lactose-free compressed tablets of the invention do readily dissolve and release their contents in vivo, e.g., in the stomach or gastrointestinal tract after being swallowed but they do not dissolve and disperse uniformly within three minutes in the DISSOLUTION TEST, rather they require more than three (3) minutes preferably more than five (5) when subjected to the DISSOLUTION TEST.

Presently, fluoxetine is not commercially sold in the United States in a tablet form. Further, tablet forms of fluoxetine hydrochloride available outside the United States are believed to be unstable due to the incorporation of lactose. See, e.g., Wirth et al., J. Pharm. Sci., 87(1):31-39 (January 1998).

In order for medicinal substances or therapeutic ingredients of the present invention (i.e., lactose-free, non-hygroscopic or anhydrous dosage forms), with or without diluents, to be made into solid dosage forms (e.g., tablets) with pressure, using available equipment, it is necessary that the material, either in crystalline or powdered form, possess a number of physical characteristics. These characteristics include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drug or therapeutic ingredient, tablets and similar dosage forms contain a number of materials referred to as additives. These additives are classified according to the role they play in the formulation of the dosage form such as a tablet, a caplet, a capsule, a troche or the like. Groups of additives include, but are not limited to, binders, diluents (fillers), disintegrants and lubricants.

While the discussion below of various additives for use in the present invention specifically refers to lactose-free dosage forms, the skilled artisan will readily understand that a subset of each category includes additives suitable for use in non-hygroscopic or anhydrous pharmaceutical compositions of the present invention. In addition, the non-hygroscopic or anhydrous pharmaceutical compositions of the present invention may also include lactose or other mono- or di-saccharides as excipients.

For non-hygroscopic formulations, special precautions must be exercised in choosing excipients and additives, such that overall, there is no propensity for moisture sorption (absorption or adsorption) in the absence of suitable environmental controls. For example, excipients for use in such formulations include, but are not limited to, alpha lactose monohydrate, mannitol and the like.

For anhydrous formulations, suitable anhydrous or low moisture forms of the below identified excipients or additives should be used, for example, AVICEL-PH-103™ and Starch 1500 LM.

A binder is used to provide a free-flowing powder from the mix of tablet ingredients so that the material will flow when used on a tablet machine. The binder also provides a cohesiveness to the lactose-free fluoxetine or its enantiomers tablet. Too little binder will give flow problems and yield tablets that do not maintain their integrity. Too much may adversely affect the release (dissolution rate) of the drug from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with the lactose-free, non-hygroscopic or anhydrous dosage formulations of fluoxetine or its enantiomers made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose or mixtures thereof.

Suitable forms of microcrystalline cellulose are, for example, the materials sold as AVICEL-PH-101 and AVICEL-PH-105 (available from FMC Corporation, American Viscose Division, Avicel Sales, Marcus Hook, Pa., U.S.A.). An exemplary suitable binder is a mixture of microcrystalline cellulose and sodium carboxymethyl cellulose sold as AVICEL RC-581 by FMC Corporation.

Most commercial tablets weigh from about 100 mg to about 500 mg total weight. Thus, for many potent drugs including lactose-free dosage forms of fluoxetine or its enantiomers, a filler may comprise a large portion of the tablet. Fillers (e.g., diluents) are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug to the filler may occur and affect bioavailability. Consequently, a sufficient amount of filler should be used to achieve a desired dilution ratio without detrimentally affecting release of the drug ingredient(s) from the dosage form containing the filler. Further, a filler that is physically and chemically compatible with the therapeutic ingredient(s) of the dosage form should be used. Thus, as noted, lactose should not be used with fluoxetine to form the lactose-free dosage forms of fluoxetine made in accordance with the present invention. It is also preferable that the lactose-free dosage forms of fluoxetine or its enantiomers according to the present invention do not include carbohydrate fillers or mono- or di-saccharides, such as, but not limited to, glucose, sucrose and fructose. The amount of filler used varies upon the type of formulation and mode of administration, and is readily discernible to those of ordinary skill in the art.

Examples of suitable fillers for use with the dosage forms made in accordance with the present invention include, but are not limited to, talc, calcium carbonate (e.g., granules or powder), dibasic calcium phosphate, tribasic calcium phosphate, calcium sulfate, (e.g., granules or powder), microcrystalline cellulose, powdered cellulose, dextrates, kaolin, mannitol, silicic acid, sorbitol, starch, pre-gelatinized starch, or mixtures thereof.

The binder/filler in pharmaceutical compositions of the present invention is typically present in about 1 to about 99 weight percent of the pharmaceutical composition.

Disintegrants are used to cause the tablet to disintegrate when exposed to an aqueous environment whether in vitro or in vivo. Too much of a disintegrant will produce tablets which may disintegrate in the bottle due to atmospheric moisture, or may cause the dosage form to begin to disintegrate in the mouth where the dosage form is exposed to either or both saliva and water or other fluids that may be taken by a patient to aid in administration. Too little may be insufficient for disintegration to occur and may thus alter the rate and extent of release of the drug ingredient(s) from the dosage form. Thus, a sufficient amount of disintegrant that is neither too little nor too much to detrimentally alter the release of the drug ingredient(s) should be used to form the dosage forms made according to the present invention. The amount of disintegrant used varies based upon the type of formulation and mode of administration, and is readily discernible to those of ordinary skill in the art. Typically, about 0.5 to about 15 weight percent of disintegrant, preferably about 1 to about 5 weight percent of disintegrant, may be used in the pharmaceutical composition.

Suitable disintegrants that may be used to form the lactose-free, non-hygroscopic or anhydrous dosage forms of fluoxetine made in accordance with the present invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof. In certain embodiments, disintegrants are specifically excluded from the composition, or are present in amounts sufficient to prevent rapid dissolving in water; for example, compressed tablets which are free of lactose are preferably non-dispersable tablets.

Based on the physicochemical properties of fluoxetine or its enantiomers, it is desirable to formulate the lactose-free, non-hygroscopic, or anhydrous pharmaceutical compositions of fluoxetine or its enantiomers such that they dissolve readily in vivo after administration to the subject, e.g., in the subject's stomach. Thus, in a preferred embodiment, the pharmaceutical compositions of the present invention include a disintegrant, such as, but not limited to, croscarmellose or sodium starch glycolate.

Whatever the dose, adhesion of the dosage form ingredients to the punches of the tableting machine must be avoided. For example, when drug (e.g., fluoxetine) accumulates on the punch surfaces, it causes the tablet surface to become pitted and therefore unacceptable. Also, sticking of drug or other dosage form ingredients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing and by the use of high levels of lubricants, e.g., magnesium stearate. However, selection of a drug salt with good anti-adhesion properties also minimizes these problems.

As noted, the lubricant is used to enhance the flow of the fluoxetine tableting powder mix to the tablet machine and to prevent sticking of the tablet in the die after the tablet is compressed. Too little lubricant will not permit satisfactory tablets to be made and too much may produce a tablet with a water-impervious hydrophobic coating. Because lubricants are usually hydrophobic materials such as stearic acid, magnesium stearate, calcium stearate and the like, a water-impervious hydrophobic coating may be formed by the use of too much lubricant. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Suitable lubricants for use with the dosage forms of fluoxetine made in accordance with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W. R. Grace Co. of Baltimore Md.), a coagulated aerosol of synthetic silica (marketed by Deaussa Co. of Plano, Tex.), CAB-O-SIL (a silicon dioxide product sold by Cabot Co. of Boston, Mass.) or mixtures thereof. A lubricant may optionally be added, typically in an amount of less than about 5 weight percent of the pharmaceutical composition.

Another class of additives for use with the dosage forms of fluoxetine, its enantiomers or salts include, but are not limited to, anti-caking agents, antimicrobial preservatives, coating agents, colorants, desiccants, flavors and perfumes, plasticizers, viscosity increasing agents, sweeteners, buffering agents, humectants and the like.

Suitable anti-caking agents for use with the dosage forms of fluoxetine, its enantiomers or salts made in accordance with the present invention include, but are not limited to, calcium silicate, magnesium silicate, silicon dioxide, colloidal silicon dioxide, talc or mixtures thereof.

Suitable antimicrobial preservatives for use with the dosage forms made in accordance with the present invention include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol or mixtures thereof.

Suitable coating agents for use with the dosage forms made in accordance with the present invention include, but are not limited to, sodium carboxymethyl cellulose, cellulose acetate phthalate, ethylcellulose, gelatin, pharmaceutical glaze, hydroxypropyl cellulose, hydroxypropyl methylcellulose (e.g., Nos.: 2208, 2906, 2910), hydroxypropyl methyl cellulose phthalate (e.g., Nos.: 200731, 220824), methylcellulose, polyethylene glycol, polyvinyl acetate phthalate, shellac, sucrose, titanium dioxide, carnauba wax, microcrystalline wax or mixtures thereof. The amount of coating agent used varies upon the type of formulation and mode of administration, and is readily discernible to those of ordinary skill in the art.

A coating of a film forming polymer may optionally be applied to the tablet (e.g., a capsule shaped tablet often referred to as a caplet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan.

In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used for immediate release tablets to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use with the present dosage formulations of fluoxetine, its enantiomers or salts include, for example, methylcellulose, hydroxypropyl methyl cellulose (PHARMACOAT 606 6 cps), polyvinylpyrrolidone (Povidone), ethylcellulose (ETHOCEL 10 cps), various derivatives of methacrylic acids and methacrylic acid esters, cellulose acetate phthalate or mixtures thereof.

Suitable colorants for use with the dosage forms of made in accordance with the present invention include, but are not limited to, pharmaceutically acceptable dyes and lakes, caramel, red ferric oxide, yellow ferric oxide or mixtures thereof. Suitable desiccants for use with the lactose-free dosage forms of fluoxetine made in accordance with the present invention include, but are not limited to, calcium chloride, calcium sulfate, silica gel or mixtures thereof.

Suitable flavors for use with the dosage forms of made in accordance with the present invention include, but are not limited to, sucrose, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Suitable plasticizers for use with the dosage forms made in accordance with the present invention include, but are not limited to, castor oil, diacetylated monoglycerides, diethyl phthalate, glycerin, mono-and di-acetylated monoglycerides, polyethylene glycol, propylene glycol, and triacetin or mixtures thereof.

Suitable viscosity increasing agents for use with the dosage forms made in accordance with the present invention include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Suitable sweetening agents for use with the dosage forms made in accordance with the present invention include, but are not limited to, aspartame, dextrates, mannitol, saccharin, saccharin calcium, saccharin sodium, sorbitol, sorbitol solution, or mixtures thereof.

Suitable buffering agents for use with the dosage forms made in accordance with the present invention include, but are not limited to, magnesium hydroxide, aluminum hydroxide and the like, or mixtures thereof. Suitable humectants include, but are not limited to, glycerol, other humectants or mixtures thereof. The dosage forms of fluoxetine may further include one or more of the following: (1) solution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g. ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

The non-hygroscopic or anhydrous dosage forms of the present invention may also be provided in the form of hard or soft capsules, for example, of gelatin or other suitable materials together with various excipients previously noted with regard to tablets. For the formation of tablets, the fluoxetine is combined with one or more excipients (e.g., diluents, binders, disintegrants, dispersing agents, surface-active agents, lubricants, coating materials, flavoring agents, coloring agents, solvents, viscosity increasing agents, suspending agents, sweeteners, colorants, dyes and the like) in various proportions using traditional tableting equipment such as twin shell or “v” blenders by known procedures to manufacture chemically and thermally stable lactose-free dosage forms (e.g., tablets, caplets and the like) containing a uniform distribution and blending of therapeutic agents. The exact amounts of each of the various excipients may be readily determined by those of ordinary skill in the pharmaceutical art.

Large-scale production of the dosage forms of fluoxetine made in accordance with the present invention requires, in addition to the therapeutic drug ingredient(s), additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like. Sugar-coating preferably does not include lactose or mono- or di-saccharides.

Tablets of the dosage forms of the present invention are typically made by molding, by compression or by generally accepted tablet forming methods. Accordingly, compressed tablets are usually prepared by large-scale production methods while molded tablets often involve small-scale operations. For example, there are three general methods of tablet preparation for making the lactose-free dosage forms of fluoxetine: (1) the wet-granulation method; (2) the dry-granulation method; and (3) direct compression. These methods are well known to those skilled in the art. See Remington's Pharmaceutical Sciences, 16th and 18th Eds., Mack Publishing Co., Easton, Pa. (1980 and 1990). See also U.S. Pharmacopeia XXI, U.S. Pharmacopeial Convention, Inc., Rockville, Md. (1985).

Various tablet formulations of the lactose-free dosage forms of fluoxetine may be made in accordance with the present invention. These include tablet dosage forms such as sugar-coated tablets, film-coated tablets, enteric-coated tablets, multiple-compressed tablets, prolonged action tablets and the like. Lactose-free, non-hygroscopic and anhydrous fluoxetine (or its enantiomers or salts) sugar-coated tablets (SCT) are compressed tablets containing a sugar coating. Such coatings may be colored and are beneficial in covering up drug substances possessing objectionable tastes or odors and in protecting materials sensitive to oxidation. Lactose-free fluoxetine film-coated tablets (FCT) are compressed tablets which are covered with a thin layer or film of a water-soluble material. A number of polymeric substances with film-forming properties may be used. The film coating imparts the same general characteristics as sugar coating with the added advantage of a greatly reduced time period required for the coating operation. Although less preferred because they delay release of the active ingredient, enteric-coated tablets are also suitable for use in the present invention. Lactose-free fluoxetine enteric-coated tablets (ECT) are compressed tablets coated with substances that resist dissolution in gastric fluid but disintegrate in the intestine. Enteric coating can be used for tablets containing drug substances which are inactivated or destroyed in the stomach, for those which irritate the mucosa or as a means of delayed release of the medication.

Lactose-free, non-hygroscopic and anhydrous forms of fluoxetine multiple compressed tablets (MCT) are compressed tablets made by more than one compression cycle such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These lactose-free fluoxetine tablets have all the advantages of compressed tablets, e.g., slotting, monogramming, in vivo disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of lactose-free fluoxetine tablets (i.e., layered tablets and press-coated tablets) may be used, for example, in the design of prolonged-action dosage forms.

Lactose-free, non-hygroscopic, or anhydrous fluoxetine prolonged-action tablets may comprise compressed tablets formulated to release the drug substance in a manner to provide medication over a period of time. There are a number of tablet types which include delayed-action tablets in which the release of the drug substance is prevented for an interval of time after administration or until certain physiological conditions exist. Repeat action tablets may be formed which periodically release a complete dose of the drug substance to the gastrointestinal fluids. Also, extended release tablets that continuously release increments of the contained drug substance to the gastrointestinal fluids may be formed.

The method of preparation and the additives to be incorporated into a lactose-free, non-hygroscopic or anhydrous tablets of the present invention are selected in order to give the tablet formulation the desirable physical characteristics allowing the rapid compression of tablets. after compression, the tablets preferably should have a number of additional attributes such as appearance, hardness, in vivo disintegration ability and uniformity which are influenced both by the method of preparation and by the additives present in the tablet formulation.

The basic unit in all tablet compression equipment includes a lower punch which fits into a die from the bottom and an upper punch, having a head of generally the same shape and dimensions as that of the lower punch, which enters the die cavity from the top after the tableting material fills the die cavity. The tablet is formed by pressure applied on the punches. Subsequently, the tablet is ejected from the die. The weight of the tablet is determined by the volume of the material which fills the die cavity.

The ability of the lactose-free, non-hygroscopic or anhydrous fluoxetine tablet or dosage form granulation to flow freely into the die cavity is important in insuring an uniform fill. The flowability of the granulation is also important to insure continuous movement of the granulation from the source of supply or feed hopper. Further, if the tablet granulation does not possess cohesive properties, after compression the tablet will crumble and fall apart on handling. Even further, as the punches must move freely within the die and the tablet must be readily ejected from the punch faces, the tableting material must have a degree of lubrication to minimize friction and to allow for the removal of the compressed tablet. A granulating agent may be added to facilitate granulation. The amount of granulating agent used varies upon the type of formulation and mode of administration, and is readily discernible to those of ordinary skill in the art. Typically, about 5 to about 15 weight percent of granulating agent is used in the pharmaceutical formulation.

Further, it is noted that stable tablets or other dosage forms thereof retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredient should not change appreciably with time.

The lactose-free pharmaceutical compositions of the present invention may also be formulated in a soft elastic gelatin capsule unit dosage form by using conventional methods, well-known in the art (see, e.g., Ebert, Pharm. Tech., 1(5):44-50 (1977)). Soft elastic gelatin capsules have a soft, globular, gelatin shell somewhat thicker than that of hard gelatin capsules, wherein a gelatin is plasticized by the addition of glycerin, sorbitol, or a similar polyol. The hardness of the capsule shell may be changed by varying the type of gelatin and the amounts of plasticizer and water. The soft gelatin shells may contain a preservative (such as methyl-and propylparabens and sorbic acid) to prevent the growth of fungi. The active ingredient may be dissolved or suspended in a liquid vehicle or carrier, such as vegetable or mineral oils, glycols such as polyethylene glycol and propylene glycol, triglycerides, surfactants such as polysorbates, or a combination thereof.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

The pharmaceutical compositions of the present invention may also be formulated so as to provide slow or controlled release of the active ingredient therein using, for example, hydroxypropylmethyl cellulose in varying proportions to provide the desired release profile, other polymer matrices, liposomes and/or microspheres.

Unless indicated otherwise, all percentages noted herein are percentages by weight based on the total weight of all the components of a particular dosage form.

A lactose-free fluoxetine dosage formulation such as a troche, a tablet or a capsule may be formed by combining the fluoxetine with one or more pharmaceutically compatible excipients in pharmaceutically compatible amounts to yield a unit dose fluoxetine dosage formulation containing from about 1 mg to about 200 mg of fluoxetine, and preferably containing from about 2 mg to about 80 mg of fluoxetine. The tablet, troche or capsule dosage formulation may be formed, for example, by methods well known in the art including wet granulation, dry granulation or compression molding. Other methods for forming tablets, troches and capsules, well known in the art, may be used. However, compression molding is preferred for the formulation of tablets and troches. For capsules, hard gelatin capsule shells are preferred which are filled with fluoxetine and one or more excipients.

STARCH 1500® is a pre-gelatinized starch manufactured by Colorcon Ltd. that is not recommended for use in amounts exceeding 75 weight percent. In addition, when magnesium stearate is used as a lubricant with STARCH 1500®, amounts greater than 0.25 weight percent of magnesium stearate should not be used, as this may have an adverse effect on dissolution. This adverse effect on dissolution in formulations of STARCH 1500® and greater than 0.25 weight percent of magnesium stearate is particularly important for compounds having relatively low water-solubility, such as fluoxetine.

Having described the invention, the following examples illustrate preferred embodiments in accordance with the presently claimed invention. It is understood that the examples are illustrative and do not limit the scope or breadth of the appended claims.

### EXAMPLE 1

### Hard Gelatin Capsule Unit Dosage Forms

| | 5 mg capsule | 10 mg capsule | 20 mg capsule |
|---|---|---|---|
| Component | (amount in mg) | (amount in mg) | (amount in mg) |
| Fluoxetine | 5.0 | 10.0 | 20.0 |
| enantiomer | | | |
| Microcrystalline | 90.0 | 90.0 | 90.0 |
| Cellulose | | | |
| Pre-gelatinized | 100.3 | 97.8 | 82.8 |
| Starch | | | |
| Croscarmellose | 7.0 | 7.0 | 7.0 |
| Magnesium | 0.2 | 0.2 | 0.2 |
| Stearate | | | |

The active ingredient is sieved and blended with the excipients listed. The mixture is filled into suitably sized two-piece hard gelatin capsules using suitable machinery and methods well known in the art. See Remington's Pharmaceutical Sciences, 16th or 18th Editions, each incorporated herein in its entirety by reference thereto. Other doses may be prepared by altering the fill weight and, if necessary, changing the capsule size to suit. Any of the stable, non-lactose hard gelatin capsule formulations above may be formed.

### EXAMPLE 2

### Compressed Tablet Formulations

| | 2.5 mg tablet | 5 mg tablet | 20 mg tablet |
|---|---|---|---|
| Component | (amount in mg) | (amount in mg) | (amount in mg) |
| Fluoxetine | 2.5 | 5.0 | 20.0 |
| Microcrystalline | 90.0 | 90.0 | 90.0 |
| Cellulose | | | |
| Pregelatinized | 100.3 | 97.8 | 82.8 |
| Starch | | | |
| Croscarmellose | 7.0 | 7.0 | 7.0 |
| Magnesium | 0.2 | 0.2 | 0.2 |
| Stearate | | | |

The active ingredient is sieved through a suitable sieve and blended with the non-lactose excipients until a uniform blend is formed. The dry blend is screened and blended with the magnesium stearate. The resulting powder blend is then compressed into tablets of desired shape and size. Tablets of other strengths may be prepared by altering the ratio of the active ingredient (i.e., fluoxetine) to the excipient(s) or modifying the tablet weight.

### EXAMPLE 3

### Wet Granulation

| | Quantity per Tablet (mg) | | |
|---|---|---|---|
| Component | Formulation A | Formulation B | Formulation C |
| Fluoxetine enantiomer | 25 | 50 | 100 |
| Pre-gelatinized starch | 100-150 | 100-125 | 50-100 |
| Microcrystalline cellu- | 0-75 | 0-50 | 0-50 |
| lose | | | |
| Povidone | 7.5 | — | 7.5 |
| Polyethylene glycol | — | 10-30 | — |
| Croscarmellose | 10 | — | 10 |
| Sodium starch glycol- | — | 5-15 | — |
| ate | | | |
| Magnesium stearate | 1.5 | 1.5 | 1.5 |
| FDC Yellow #2 lake | 1.25 | 1.25 | 1.25 |

The active ingredient is sieved through a suitable screen and blended with the non-lactose excipients (excluding half of the croscarmellose (or sodium starch glycolate) and all of the microcrystalline cellulose) until a uniform blend is formed. Suitable volumes of water are added and the powder granulated. After drying, the granules are screened and blended with the microcrystalline cellulose, the remainder of croscarmellose or sodium starch glycolate, and briefly with the magnesium stearate. The resulting free-flowing powder is then compressed into tablets of desired shape and size. Tablets of other strengths may be prepared by altering the ratio of the active ingredient (i.e., fluoxetine) to the excipients or modifying the tablet weight.

### EXAMPLE 4

### Direct Compression

| | Quantity per Tablet (mg) | |
|---|---|---|
| Component | Formulation A | Formulation B |
| Fluoxetine | 25 | 50 |
| Pre-gelatinized starch | 12.5 | 12.5 |
| Microcrystalline cellulose | 205 | 180 |
| Silicon dioxide | 0.625 | 0.625 |
| Sodium lauryl sulfate | 1.25 | 1.25 |
| Croscarmellose | 2.5 | 2.5 |
| Magnesium stearate | 2 | 2 |
| FDC Yellow #2 lake | 1.25 | 1.25 |

The active ingredient is passed through a suitable sieve and blended with the non-lactose excipients (except magnesium stearate) until a uniform blend is formed. The dry blend is screened and blended briefly with magnesium stearate. The resulting powder blend is then compressed into tablets of desired shape and size. Tablets of other strengths may be prepared by altering the ratio of the active ingredient (i.e., fluoxetine) to the excipients or modifying the tablet weight.

While the present invention has been described with respect to the particular embodiments, it will be apparent to those skilled in the art that various changes and modifications may be made without departing from the spirit and scope of the invention as defined in the claims.

What is claimed is:

1. A lactose-free pharmaceutical composition which comprises an optically pure enantiomer of fluoxetine, or a pharmaceutically acceptable salt thereof, and at least one non-lactose pharmaceutically acceptable excipient.

2. A solid pharmaceutical composition which comprises an optically pure enantiomer of fluoxetine, or a pharmaceutically acceptable salt thereof, and a pharmaceutically acceptable excipient, wherein said excipient is not lactose.

3. The composition of claim 1, wherein said non-lactose pharmaceutically acceptable excipient is a binder, a filler, or a mixture thereof.

4. The composition of claim 2, wherein said pharmaceutically acceptable excipient is a binder, a filler, or a mixture thereof.

5. The composition of claim 3 or 4 wherein said binder is a starch.

6. The composition of claim 3 or 4 wherein said binder is a cellulose.

7. The composition of claim 5 wherein said starch is selected from the group consisting of corn starch, potato starch, pre-gelatined starch and a mixture thereof.

8. The composition of claim 6 wherein said cellulose is selected from the group consisting of ethyl cellulose, cellulose acetate, carboxymethyl cellulose calcium, sodium carboxymethyl cellulose, methyl cellulose, hydroxypropyl methyl cellulose, microcrystalline cellulose and a mixture thereof.

9. The composition of claim 3 or 4, which further comprises a lubricant, disintegrant, or mixtures thereof.

10. The composition of claim 1 or 2, wherein said enantiomer of fluoxetine is (R)-fluoxetine.

11. The composition of claim 1 or 2, wherein said enantiomer of fluoxetine is (S)-fluoxetine.

12. The composition of claim 1 or 2, wherein said pharmaceutical composition is substantially free of all mono- or di-saccharides.

13. A chemically stable compressed tablet free of lactose which comprises racemic fluoxetine, an optically pure enantiomer of fluoxetine or a pharmaceutically acceptable salt thereof, and at least one pharmaceutically acceptable excipient.

14. A chemically stable compressed tablet free of lactose which comprises about 1% to about 50% by weight of racemic fluoxetine, an optically pure enantiomer or a pharmaceutically acceptable salt thereof, and about 99% to about 50% by weight of at least one pharmaceutically acceptable excipient.

15. The compressed tablet of claims 13 or 14 wherein said tablet does not contain a disintegrant.

16. The compressed tablet of claim 13 or 14 wherein said tablet does not dissolve in less than three minutes when subjected to the DISSOLUTION TEST.

17. The composition of claim 13 or 14, wherein said fluoxetine is present in an amount from about 1 mg to about 200 mg.

18. The composition of claim 17, wherein said fluoxetine is present in an amount of about 2 mg to about 100 mg.

19. The composition of claim 13 or 14, wherein said fluoxetine enantiomer is optically pure (R)-fluoxetine.

20. The composition of claim 13 or 14, wherein said fluoxetine enantiomer is optically pure (S)-fluoxetine.

21. A solid compressed tablet consisting essentially of racemic fluoxetine, an optically pure enantiomer or a pharmaceutically acceptable salt thereof, and microcrystalline cellulose and pre-gelatinized starch.

22. The solid pharmaceutical composition of claim 13 or 14, wherein said compressed tablet is sterile, anhydrous and non-hygroscopic.

23. An anhydrous solid pharmaceutical composition which comprises racemic fluoxetine, an optically pure enantiomer of racemic fluoxetine or a pharmaceutically acceptable salt thereof, and one or more pharmaceutically acceptable excipients.

24. The composition of claim 23 wherein said composition does not contain lactose.

25. The composition of claim 23 or 24 wherein said composition is a compressed tablet.

26. The composition of claim 23 or 24 wherein said fluoxetine enantiomer is optically pure (R)-fluoxetine.

27. The composition of claim 23 or 24 wherein said fluoxetine enantiomer is optically pure (S)-fluoxetine.

28. The composition of claim 23 or 24 wherein said composition is non-hygroscopic.

29. The composition of claim 1, 13, 14, 21, 23, or 24 wherein said pharmaceutically acceptable salt is a hydrochloride salt.

30. A stable solid pharmaceutical unit dosage form which comprises racemic fluoxetine, an optically pure enantiomer of racemic fluoxetine, or a pharmaceutically acceptable salt thereof, and one or more pharmaceutically acceptable excipients wherein said dosage form is not a capsule or gel cap.

31. The unit dosage form of claim 30 wherein said fluoxetine enantiomer is optically pure (R)-fluoxetine.

32. The unit dosage form of claim 30 wherein said fluoxetine enantiomer is optically pure (S)-fluoxetine.

33. A solid compressed tablet substantially free of lactose which comprises an optically pure enantiomer of fluoxetine, or a pharmaceutically acceptable salt thereof, and at least one pharmaceutically acceptable excipient which is not lactose.

34. A disintegrating tablet substantially free of lactose which comprises an optically pure enantiomer of fluoxetine, or a pharmaceutically acceptable salt thereof, and at least one pharmaceutically acceptable excipient which is not lactose.

35. A method of treating depression in a mammal which comprises the oral administration of a therapeutically effective amount of a composition of claims 1, 2, 13, 14, 21, 23, 24, 30, 33 or 34 to said mammal.
